(19) United States
(12) Patent Application Publication
(10) Pub. No.: US 2022/0296679 A1
(43) Pub. Date: Sep. 22, 2022

(54) TREATMENT OF NEUROPATHIC PAIN ASSOCIATED WITH CHEMOTHERAPY-INDUCED PERIPHERAL NEUROPATHY

(71) Applicant: Helixmith Co., Ltd, Seoul (KR)

(72) Inventors: Junghun LEE, Seoul (KR); Nayeon LEE, Seoul (KR)

(21) Appl. No.: 17/523,740

(22) Filed: Nov. 10, 2021

Related U.S. Application Data
(63) Continuation of application No. 16/414,115, filed on May 16, 2019, now abandoned.
(60) Provisional application No. 62/673,048, filed on May 17, 2018.

(51) Int. Cl. A61K 38/18; A61P 23/00; A61K 9/00; A61K 31/7088

(52) U.S. Cl. CPC A61K 38/1833; A61P 23/00; A61K 9/0019; A61K 31/7088

(57) ABSTRACT

The present invention relates to methods of treating chemotherapy-induced peripheral neuropathy. In particular, the methods provide a new way of reducing neuropathic pain associated with chemotherapy-induced peripheral neuropathy by administering a nucleic acid construct encoding human HGF proteins. This application further provides nucleic acid constructs, pharmacological compositions, and methods of administration of the nucleic acid constructs that are effective in treating the neuropathic pain.

### 1. CROSS REFERENCE TO RELATED APPLICATION

This application is a continuation of co-pending U.S. application Ser. No. 16/414,115, filed May 16, 2019, which claims the benefit of and priority to U.S. Provisional Application No. 62/673,048, filed on May 17, 2018, each of which is incorporated herein by reference in its entirety for all purposes.

### 2. SEQUENCE LISTING

The instant application contains a Sequence Listing which has been submitted via EFS-Web and is hereby incorporated by reference in its entirety. Said ASCII copy, created on October 23, 2021, is named 50209US CRF sequencelisting.txt, and is 80,274 bytes in size.

### 3. BACKGROUND

Chemotherapy-induced peripheral neuropathy (CIPN) is a common side effect of certain cancer treatments and has a significant impact on patients' long-term quality of life. Symptoms of CIPN include unusual sensations (paresthesia), numbness, balance problems, and pain. Specific symptoms vary depending on the type of chemotherapy administered, but patients suffering from CIPN typically have a high risk of developing neuropathic pain.

Chemotherapeutic agents that are known to cause CIPN and associated pain include platinum analogs, antitubulins (taxanes, vinca alkaloids, eribulin), proteasome inhibitors (bortezomib, carfilzomib), immunomodulatory agents (thalidomide, lenalidomide, pomalidomide), and even some of the newer biologics that are not conventionally considered to be chemotherapeutic agents (alemtuzumab, ipilimumab, brentuximab). Some of these agents, e.g. oxaliplatin, cisplatin, and vincristine, are further known to induce symptoms that continue to progress even after the treatment has ended.

One of the main challenges in managing CIPN is that the exact pathophysiology is not well understood. Furthermore, various chemotherapeutic agents are likely to cause CIPN by different pathophysiological mechanisms. Despite continuing efforts to elucidate the exact pathophysiology, clinically relevant therapeutic interventions are not available.

Neuropathic pain associated with CIPN has been managed in a manner similar to other types of neuropathic pain—that is, with a combination of physical therapy, complementary therapies such as massage and acupuncture, and medications. Various medications have been used or suggested for use, such as gabapentin, pregabalin, carbamazepine, tricyclic antidepressants, oxycodone, morphine, methadone, tramadol, duloxetine, and venlafaxine. However, none of these therapies has demonstrated true efficacy in reducing the pain of CIPN, and the medications have side effects of their own.

Recently, Kessler and colleagues reported a successful double-blind, placebo-controlled, phase 2 human clinical trial of nonviral HGF gene therapy in diabetic peripheral neuropathy. Kessler et al., Annals Clin. Transl. Neurology 2(5):4650478 (2015). Injection of the plasmid VM202 (pCK-HGF-X7), which expresses two isoforms of human HGF, into the calf muscle of patients with diabetic peripheral neuropathy significantly reduced pain, with two days of treatment sufficient to provide symptomatic relief with improvement in quality of life for 3 months. However, this therapy has not yet been shown to be effective in reducing the pain of CIPN.

Therefore, there is a need to develop an effective drug for treating pain associated with CIPN, whose etiology and pathophysiological mechanisms have not been fully understood. There is a particular need to assess whether VM202 (pCK-HGF-X7) can be effective in reducing the pain of CIPN.

### 4. SUMMARY

Some aspects of the present invention relate to methods of treating neuropathic pain associated with exposure to a neuropathy-inducing therapeutic agent by administering a nucleic acid construct encoding a human HGF protein.

The methods can comprise the steps of administering to a subject that has been previously exposed to the therapeutic agent a first therapeutically effective amount of a nucleic acid construct encoding two isoforms of a human HGF protein, wherein the nucleic acid construct comprises: a first sequence comprising exons 1-4 of a human HGF gene or a degenerate sequence of the first sequence, a second sequence comprising intron 4 of the human HGF gene or a fragment of the second sequence, and a third sequence comprising exons 5-18 of the human HGF gene or a degenerate sequence of the third sequence.

In some embodiments, the neuropathy-inducing therapeutic agent is a chemotherapy drug. The chemotherapy drug is selected from the group consisting of a plant alkaloid, a taxane, an epothilone, a proteasome inhibitor, an immunomodulator, and an antineoplastic biologic. In some embodiments, the chemical drug is vincristine, bortezomib, paclitaxel, or cisplatin.

In some embodiments, the subject is a human patient. In some embodiments, the subject has cancer.

In some embodiments, the method further comprises the step of readministering the nucleic acid construct to the subject more than one week after the step of administering the first therapeutically effective amount of nucleic acid construct. In some embodiments, the step of readministering is done at least 2 weeks, 3 weeks, 4 weeks, 5 weeks, or 10 weeks after the step of administering the first therapeutically effective amount of nucleic acid construct. In some embodiments, the step of readministering is done at least 10 days, 15 days, 20 days, 30 days, 40 days, 50 days or 100 days after the step of administering the first therapeutically effective amount of nucleic acid construct. In some embodiments, the subject is not administered with the nucleic acid construct between the step of administering the first therapeutically effective amount of nucleic acid construct and the step of readministering.

In some embodiments, the first sequence and the third sequence are devoid of an intron. In some embodiments, the two isoforms of HGF comprise a full-length HGF (flHGF) and a deleted variant HGF (dHGF). The full-length HGF (flHGF) can comprise a polypeptide of SEQ ID NO:1 and the deleted variant HGF (dHGF) can comprise a polypeptide of SEQ ID NO:2.

In some embodiments, the first sequence comprises a polynucleotide of SEQ ID NO:3. In some embodiments, the second sequence comprises a polynucleotide of SEQ ID NO:6. In some embodiments, the third sequence comprises a polynucleotide of SEQ ID NO:4.

In some embodiments, the nucleic acid construct comprises a polynucleotide of SEQ ID NO:13. In some embodiments, the nucleic acid construct further comprises a pCK vector.

In some embodiments, the step of administering the first therapeutically effective amount of nucleic acid construct or the step of readministering comprises one or more intramuscular injections of the nucleic acid construct. In some embodiments, the first therapeutically effective amount of nucleic acid construct is between 1 μg and 100 mg, between 10 μg and 50 mg, between 100 μg and 10 mg, between 1 mg and 25 mg, or between 1 mg and 10 mg.

Some other aspect of the present invention relates to a nucleic acid construct encoding a human HGF for treating neuropathic pain associated with exposure to a neuropathy-inducing therapeutic agent. The nucleic acid construct can comprise: a first sequence comprising exons 1-4 of a human HGF gene or a degenerate sequence of the first sequence, a second sequence comprising intron 4 of the human HGF gene or a fragment of the second sequence, and a third sequence comprising exons 5-18 of the human HGF gene or a degenerate sequence of the third sequence. In other aspects, the present invention provides a pharmaceutical composition comprising a nucleic acid construct encoding a human HGF for treating neuropathic pain associated with exposure to a neuropathy-inducing therapeutic agent.

### 5. BRIEF DESCRIPTION OF THE DRAWINGS

FIG. 1 illustrates the structure of a nucleic acid construct, pCK-HGF-X7, also known as VM202. The pCK vector comprises (1) the promoter/enhancer and 5′ UTR (exon 1, intron A and partial exon 2) derived from HCMV IE gene (“HCMV IE promoter”), (2) a ColE1 origin of replication (“ColE1”), and (3) Kanamycin resistance gene (“Kan^{t}”). The HGF-X7 insert (“HGF-X7”) is a cDNA containing exons 1-18 of human HGF and a fragment of intron 4 of the human HGF gene. A sequence element fused in-frame with the 3′ end of the HGF-X7 insert encodes a poly-A tail (“pA”). pCK-HGF-X7 expresses both HGF₇₂₃ (dHGF) and HGF₇₂₈ (flHGF) via alternative splicing.

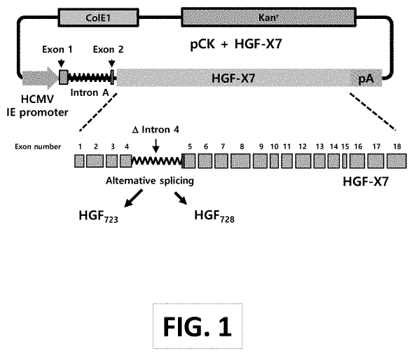

FIG. 2A outlines an experimental procedure for testing the effects of pCK-HGF-X7 on paclitaxel-induced neuropathic pain. Specifically, 9-week old Balb/c female mice were administered with 1 mg/kg paclitaxel for 1 week on a daily basis via intraperitoneal injection. 200 μg of plasmid DNAs, pCK or pCK-HGF-X7 was injected intramuscularly in week 1. The severity of pain symptom was determined by examining mechanical allodynia using Von Frey's filament every week. FIG. 2B provides data obtained from the experiment outlined in FIG. 2A. Specifically, FIG. 2B provides paw withdrawal response (frequency (%)) data measured in the mice administered paclitaxel. The paw withdrawal frequency decreased significantly in the group administered pCK-HGF-X7, but not in the control group administered with pCK vector lacking the HGF-X7 insert.

FIG. 3A outlines an experimental procedure for testing the effects of pCK-HGF-X7 on vincristine-induced neuropathic pain. Specifically, 5-week old Balb/c male mice were administered 200 μg/kg vincristine for two weeks on a daily basis through i.p. injection and administered 200 μg pCK-HGF-X7 in week 1. Their pain level was determined by Von Frey's Filament test every week. FIG. 3B provides data obtained from the experiment outlined in FIG. 3A. Specifically, FIG. 3B provides paw withdrawal response (frequency (%)) data measured in the mice administered vincristine. The paw withdrawal frequency decreased significantly in the group administered pCK-HGF-X7, but not in the control group administered with pCK vector lacking the HGF-X7 insert.

FIG. 4A outlines an experimental procedure for testing the effects of pCK-HGF-X7 on bortezomib-induced neuropathic pain. Specifically, 7-week old C57BL6 male mice were administered 0.4 mg/kg bortezomib three times a week for two weeks by i.p. injections and administered 200 μg pCK-HGF-X7 in week 2. Their pain level was determined by Von Frey's Filament test every week. FIG. 4B provides data obtained from the experiment outlined in FIG. 4A. Specifically, FIG. 4B provides paw withdrawal response (frequency (%)) data measured in the mice administered bortezomib. The paw withdrawal frequency decreased significantly in the group administered pCK-HGF-X7, but not in the control group administered with pCK vector lacking the HGF-X7 insert.

FIG. 5A outlines an experimental procedure for testing the effects of pCK-HGF-X7 on cisplatin-induced neuropathic pain. Specifically, 9-week old C57BL6 male mice were administered 2.3 mg/kg cisplatin once every two days for two weeks by i.p. injections and administered 200 μg pCK-HGF-X7 in week 1. Their pain level was determined by Von Frey's Filament test every week. FIG. 5B provides data obtained from the experiment outlined in FIG. 5A. Specifically, FIG. 5B provides paw withdrawal response (frequency (%)) data measured in the mice administered cisplatin. The paw withdrawal frequency decreased significantly in the group administered pCK-HGF-X7, but not in the control group administered with pCK vector lacking the HGF-X7 insert.

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

The term “isoforms of HGF” as used herein refers to a polypeptide having an amino acid sequence that is at least 80% identical to the amino acid sequence of a naturally occurring HGF polypeptide in an animal. The term includes polypeptides having an amino acid sequence that is at least 80% identical to any full length wild type HGF polypeptide, and includes polypeptides having an amino acid sequence that is at least 80% identical to a naturally occurring HGF allelic variant, splice variant, or deletion variant. Isoforms of HGF preferred for use in the present invention include two or more isoforms selected from the group consisting of full-length HGF (flHGF) (synonymously, fHGF), deleted variant HGF (dHGF), NK1, NK2, and NK4. According to a more preferred embodiment of the present invention, the isoforms of HGF used in the methods described herein include flHGF and dHGF.

The terms “human f1HGF”, “f1HGF” and “fHGF” are used interchangeably herein to refer to a protein consisting of amino acids 1-728 of the human HGF protein. The sequence of flHGF is provided in SEQ ID NO: 1.

The terms “human dHGF” and “dHGF” are used interchangeably herein to refer to a deleted variant of the HGF protein produced by alternative splicing of the human HGF gene. Specifically, “human dHGF” or “dHGF” refers to a human HGF protein with deletion of five amino acids (F, L, P, S, and S) in the first kringle domain of the alpha chain from the full length HGF sequence. Human dHGF is 723 amino acids in length. The amino acid sequence of human dHGF is provided in SEQ ID NO: 2.

The term “treatment” as used herein refers to all the acts of (a) suppressing neuropathic pain; (b) alleviation of neuropathic pain; and (c) removal of neuropathic pain. In some embodiments, the composition of the present invention can treat neuropathic pain through the growth of neuronal cells or the suppression of neuronal cell death.

The term “therapeutically effective dose” or “effective amount” as used herein refers to a dose or amount that produces the desired effect for which it is administered. In the context of the present methods, a therapeutically effective amount is an amount effective to reduce neuropathic pain associated with CIPN.

The term “sufficient amount” as used herein refers to an amount sufficient to produce a desired effect.

The term “degenerate sequence” as used herein refers to a nucleic acid sequence that can be translated to provide an amino acid sequence identical to that translated from the reference nucleic acid sequence.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 6.3. Methods of Treating Neuropathic Pain Associated with CIPN

In a first aspect, methods are presented for treating neuropathic pain associated with chemotherapy-induced peripheral neuropathy. In typical embodiments, the methods comprise administering to a subject that has been exposed to a therapeutic agent that induces peripheral neuropathy a therapeutically effective amount of a nucleic acid construct that expresses two isoforms of a human HGF protein.

### 6.3.1. Nucleic Acid Construct Expressing Two Hepatocyte Growth Factor (HGF) Isoforms

In the methods described herein, the nucleic acid construct expresses at least two isoforms of a human HGF protein. In some embodiments, the nucleic acid construct expresses two isoforms. In typical embodiments, the nucleic acid construct expresses at least one of flHGF and dHGF. In particular embodiments, the nucleic acid construct expresses both flHGF and dHGF.

flHGF and dHGF share several biological functions, but differ in terms of immunological characteristics and several biological properties. For example, flHGF exhibits about 20-fold, 10-fold and 2-fold higher activities than dHGF in promoting DNA synthesis in human umbilical cord venous endothelial cell, arterial smooth muscle cell, and NSF-60 (murine myeloblast cell), respectively. On the other hand, dHGF exhibits about 3-fold and 2-fold higher activities than flHGF in promoting DNA synthesis of LLC-PK1 (pig kidney epithelial cells), and OK (American opossum kidney epithelial cells), and mouse interstitial cells, respectively. In addition, flHGF exhibits 70-fold higher solubility in PBS than dHGF. Several anti-dHGF monoclonal antibodies recognize only dHGF, which implies that the three-dimensional structures of flHGF and dHGF are different.

### 6.3.1.1. Expressed Sequences

In some embodiments, the construct expresses two or more isoforms of HGF by comprising an expression regulatory sequence for each isoform coding sequence (CDS). In some embodiments, the construct comprises an internal ribosomal entry site (IRES) between two coding sequences, for example, in the order of (1) expression regulatory sequence-(2) coding sequence of first isomer-(3) IRES-(4) coding sequence of second isomer-(5) transcription termination sequence. IRES allows translation to start at the IRES sequence, thereby allowing expression of two genes of interest from a single construct. In yet further embodiments, a plurality of constructs, each encoding a single isoform of HGF, are used together to induce expression of more than one isoforms of HGF in the subject to whom administered.

Preferred embodiments of the methods of the present invention use a construct that simultaneously expresses two or more different types of isoforms of HGF—i.e., flHGF and dHGF—by comprising an alternative splicing site. It was previously demonstrated in U.S. Pat. No. 7,812,146, incorporated by reference herein, that a construct encoding two isoforms of HGF (flHGF and dHGF) through alternative splicing has much higher (almost 250 fold higher) expression efficiency than a construct encoding one isoform of HGF (either flHGF or dHGF). In typical embodiments, the construct comprises (i) a first sequence comprising exons 1-4 of a human HGF gene or a degenerate sequence of the first sequence; (ii) a second sequence comprising intron 4 of the human HGF gene or a fragment of the second sequence; and (iii) a third sequence comprising exons 5-18 of the human HGF gene or a degenerate sequence of the third sequence. From the construct, two isoforms of HGF (flHGF and dHGF) can be generated by alternative splicing between exon 4 and exon 5.

In some embodiments, the construct comprises a full sequence of intron 4. In some embodiments, the construct comprises a fragment of intron 4. In preferred embodiments, the construct comprises a nucleotide sequence selected from the group consisting of SEQ ID NO: 7 to SEQ ID NO: 14. The nucleotide sequence of SEQ ID NO:7 is 7113 bp and corresponds to a construct comprising the full sequence of intron 4. The nucleotide sequence of SEQ ID NOS: 8-14 correspond to constructs comprising various fragments of intron 4.

Various fragments of intron 4 can be inserted between exon 4 and exon 5 to induce expression of both flHGF and dHGF. For example, (i) nucleotides 483-2244 and nucleotides 3168-5438 of SEQ ID NO: 7; (ii) nucleotides 483-2244 and nucleotides 4168-5438 of SEQ ID NO: 7; (iii) nucleotides 483-2244 and nucleotides 5117-5438 of SEQ ID NO: 7; (iv) nucleotides 483-728 and nucleotides 2240-5438 of SEQ ID NO: 7. (v) nucleotides 483-728 and nucleotides 3168-5438 of SEQ ID NO: 7; (vi) nucleotides 483-728 and nucleotides 4168-5438 of SEQ ID NO: 7, or (vii) nucleotides 483-728 and nucleotides 5117-5438 of SEQ ID NO: 7 can be used.

Thus, constructs used in the methods of the present invention can comprise: (i) (exon 1 to exon 4)-(nucleotides 483-5438 of SEQ ID NO: 7)-(exon 5 to exon 18); (ii) (exon 1 to exon 4)-(nucleotides 483-2244 nucleotides 3168-5438 of SEQ ID NO: 7)-(exon 5 to exon 18); (iii) (exon 1 to exon 4)-(nucleotides 483-2244 nucleotides 4168-5438 of SEQ ID NO: 7)-(exon 5 to exon 18); (iv) (exon 1 to exon 4)-(nucleotides 483-2244 nucleotides 5117-5438 of SEQ ID NO: 7)-(exon 5 to exon 18) ; (v) (exon 1 to exon 4)-(nucleotides 483-728 nucleotides 2240-5438 of SEQ ID NO: 7)-(exon 5 to exon 18); (vi) (exon 1 to exon 4)-(nucleotides 483-728 nucleotides 3168-5438 of SEQ ID NO: 7)-(exon 5 to exon 18); (vii) (exon 1 to exon 4)-(nucleotides 483-728 nucleotides 4168-5438 of SEQ ID NO: 7)-(exon 5 to exon 18); or (viii) (exon 1 to exon 4)-(nucleotides 483-728 nucleotides 5117-5438 of SEQ ID NO: 7)-(exon 5 to exon 18).

Various nucleic acid constructs comprising cDNA corresponding exon 1-18 of human HGF and intron 4 of a human HGF gene or its fragment are named “HGF-X” followed by a unique number. The HGF-X generated and tested by Applicant includes, but not limited to, HGF-X1, HGF-X2, HGF-X3, HGF-X4, HGF-X5, HGF-X6, HGF-X7, and HGF-X8 having nucleotide sequences of SEQ ID NO: 7 to SEQ ID NO: 14, as summarized below in TABLE 1.

TABLE 1

| | | | Sequence of Intron |
|---|---|---|---|
| Name | Structure | Sequence | between Exons 4 and 5 |
| HGF-X1 | (exon 1 to exon 4)-(full | SEQ ID NO: 7 | nucleotides 483-5438 of SEQ |
| | sequence of intron 4)-(exon 5 | | ID NO: 7 |
| | to exon 18) | | |
| HGF-X2 | (exon 1 to exon 4)-(fragment | SEQ ID NO: 8 | nucleotides 483-2244 and |
| | of intron 4)-(exon 5 to exon | | nucleotides 3168-5438 of |
| | 18) | | SEQ ID NO: 7. |
| HGF-X3 | (exon 1 to exon 4)-(fragment | SEQ ID NO: 9 | nucleotides 483-2244 and |
| | of intron 4)-(exon 5 to exon | | nucleotides 4168-5438 of |
| | 18) | | SEQ ID NO: 7. |
| HGF-X4 | (exon 1 to exon 4)-(fragment | SEQ ID NO: 10 | nucleotides 483-2244 and |
| | of intron 4)-(exon 5 to exon | | nucleotides 5117-5438 of |
| | 18) | | SEQ ID NO: 7. |
| HGF-X5 | (exon 1 to exon 4)-(fragment | SEQ ID NO: 11 | nucleotides 483-728 and |
| | of intron 4)-(exon 5 to exon | | nucleotides 2240-5438 of |
| | 18) | | SEQ ID NO: 7. |
| HGF-X6 | (exon 1 to exon 4)-(fragment | SEQ ID NO: 12 | nucleotides 483-728 and |
| | of intron 4)-(exon 5 to exon | | nucleotides 3168-5438 of |
| | 18) | | SEQ ID NO: 7. |
| HGF-X7 | (exon 1 to exon 4)-(fragment | SEQ ID NO: 13 | nucleotides 483-728 and |
| | of intron 4)-(exon 5 to exon | | nucleotides 4168-5438 of |
| | 18) | | SEQ ID NO: 7. |
| HGF-X8 | (exon 1 to exon 4)-(fragment | SEQ ID NO: 14 | nucleotides 483-728 and |
| | of intron 4)-(exon 5 to exon | | nucleotides 5117-5438 of |
| | 18) | | SEQ ID NO: 7. |

Applicant previously has demonstrated that HGF-X7 showed the highest expression efficiency as disclosed in U.S. Pat. No. 7,812,146, incorporated by reference in its entirety herein. Accordingly, a nucleic acid construct comprising HGF-X7 can be used in preferred embodiments of the methods of the present invention.

The amino acid sequences and nucleotide sequences of HGF isoforms used in this invention may further include amino acid sequences and nucleotide sequences substantially identical to sequences of the wild type human HGF isoforms. The substantial identity includes sequences with at least 80% identity, more preferably at least 90% identity and most preferably at least 95% identity where the amino acid sequence or nucleotide sequence of the wild type human HGF isoform is aligned with a sequence in the maximal manner. Methods of alignment of sequences for comparison are well-known in the art. Various programs and alignment algorithms are described in: Smith and Waterman, Adv. Appl. Math. 2: 482 (1981); Needleman and Wunsch, J. Mol. Bio. 48: 443 (1970); Pearson and Lipman, Methods in Mol. Biol. 24: 307-31 (1988); Higgins and Sharp, Gene 73: 15 237-44 (1988); Higgins and Sharp, CABIOS 5: 151-3 (1989) Corpet et al., Nuc. Acids Res. 16: 10881-90 (1988); Huang et al., Comp. Appl. BioSci. 8: 155-65 (1992); and Pearson et al., Meth. Mol. Biol. 24: 307-31 (1994). The NCBI Basic Local Alignment Search Tool (BLAST) [Altschul 20 et al., J. Mol. Biol. 215: 403-10 (1990) J is available from several sources, including the National Center for Biological Information (NBCl, Bethesda, Md.) and on the Internet, for use in connection with the sequence analysis programs blastp, blasm, blastx, tblastn and tblastx. BLAST and a description of how to determine sequence identify using the program can be accessed at the official website of NCBI (National Center for Biotechnology Information) under NIH (National Institute of Health).

### 6.3.1.2. Vector

Constructs used in the methods of the present invention typically comprise a vector with one or more regulatory sequences (e.g., a promoter or an enhancer) operatively linked to the expressed sequences. The regulatory sequence regulates expression of the isoforms of HGF.

It is preferred that the polynucleotide encoding one or more isoforms of HGF proteins is operatively linked to a promoter in an expression construct. The term “operatively linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

In typical embodiments, the promoter linked to the polynucleotide is operable in, preferably, animal, more preferably, mammalian cells, to control transcription of the polynucleotide, including the promoters derived from the genome of mammalian cells or from mammalian viruses, for example, CMV (cytomegalovirus) promoter, the adenovirus late promoter, the vaccinia virus 7.5K promoter, SV40 promoter, HSV tk promoter, RSV promoter, EF1 alpha promoter, metallothionein promoter, beta-actin promoter, human IL-2 gene promoter, human IFN gene promoter, human IL-4 gene promoter, human lymphotoxin gene promoter and human GM-CSF gene promoter, but not limited to those. More preferably, the promoter useful in this invention is a promoter derived from the IE (immediately early) gene of human CMV (hCMV) or EF1 alpha promoter, most preferably hCMV IE gene-derived promoter/enhancer and 5′ -UTR (untranslated region) comprising the overall sequence of exon 1 and exon 2 sequence spanning a sequence immediately before the ATG start codon.

The expression cassette used in this invention may comprise a polyadenylation sequence, for example, including bovine growth hormone terminator (Gimmi, E. R., et al., Nucleic Acids Res. 17:6983-6998 (1989)), SV40- derived polyadenylation sequence (Schek, N, et al., Mol. Cell Biol. 12:5386-5393 (1992)), HIV-1 polyA (Klasens, B. I. F., et al., Nucleic Acids Res. 26:1870-1876 (1998)), β-globin polyA (Gil, A., et al, Cell 49:399-406 (1987)), HSV TK polyA (Cole, C. N. and T. P. Stacy, Mol. Cell. 5 Biol. 5: 2104-2113 (1985)) or polyoma virus polyA (Batt, D. Band G. G. Carmichael, Mol. Cell. Biol. 15:4783-4790 (1995)), but not limited thereto.

### 6.3.1.2.1. Non-Viral Vector

In some embodiments, the nucleic acid construct is a non-viral vector capable of expressing two or more isoforms of HGF.

In typical embodiments, the non-viral vector is a plasmid. In currently preferred embodiments, the plasmid is pCK, pCP, pVAX1 or pCY. In particularly preferred embodiments, the plasmid is pCK, details of which can be found in WO 2000/040737 and Lee et al., Biochem. Biophys. Res. Comm. 272:230-235 (2000), both of which are incorporated herein by reference in their entireties. The pCK vector has a polynucleotide of SEQ ID NO:5. E. coli transformed with pCK was deposited at the Korean Culture Center of Microorganisms (KCCM) under the terms of the Budapest Treaty on Mar. 21, 2003 (Accession No: KCCM-10476).

In particularly preferred embodiments, the pCK plasmid containing the HGF-X7 expression sequences is used as the nucleic acid construct in the methods of the present invention. One preferred embodiment, pCK-HGF-X7 (also called VM202), has been deposited (in the form of an E. coli strain transformed with the plasmid) under the terms of the Budapest Treaty at the KCCM under accession number KCCM-10361.

### 6.3.1.2.2. Viral Vector

In other embodiments, various viral vectors known in the art can be used to deliver and express one or more isoforms of HGF proteins of the present invention. For example, vectors developed using retroviruses, lentiviruses, adenoviruses, or adeno-associated viruses can be used for some embodiments of the present invention.

### (a) Retrovirus

Retroviruses capable of carrying relatively large exogenous genes have been used as viral gene delivery vectors in the senses that they integrate their genome into a host genome and have broad host spectrum.

In order to construct a retroviral vector, the polynucleotide of the invention is inserted into the viral genome in the place of certain viral sequences to produce a replication-defective virus. To produce virions, a packaging cell line containing the gag, pol and env genes but without the LTR (long terminal repeat) and W components is constructed (Mann et al., Cell, 33:153-159(1983)). When a recombinant plasmid containing the polynucleotide of the invention, LTR and W is introduced into this cell line, the W sequence allows the RNA transcript of the recombinant plasmid to be packaged into viral particles, which are then secreted into the culture media (Nicolas and Rubinstein “Retroviral vectors,” In: Vectors: A survey of molecular cloning vectors and their uses, Rodriguez and Denhardt (eds.), Stoneham: Butterworth, 494-513(1988)) The media containing the recombinant retroviruses is then collected, optionally concentrated and used for gene delivery.

A successful gene transfer using the second generation retroviral vector has been reported. Kasahara et al. (Science, 266:1373-1376 (1994)) prepared variants of moloney murine leukemia virus in which the EPO (erythropoietin) sequence is inserted in the place of the envelope region, consequently, producing chimeric proteins having novel binding properties. Likely, the present gene delivery system can be constructed in accordance with the construction strategies for the second-generation retroviral vector.

### (b) Lentiviruses

Lentiviruses can be also used in some embodiments of the present invention. Lentiviruses are a subclass of Retroviruses. However, Lentivirus can integrate into the genome of non-dividing cells, while Retroviruses can infect only dividing cells.

Lentiviral vectors are usually produced from packaging cell line, commonly HEK293, transformed with several plasmids. The plasmids include (1) packaging plasmids encoding the virion proteins such as capsid and the reverse transcriptase, (2) a plasmid comprising an exogenous gene to be delivered to the target.

When the virus enters the cell, the viral genome in the form of RNA is reverse-transcribed to produce DNA, which is then inserted into the genome by the viral integrase enzyme. Thus, the exogenous delivered with the Lentiviral vector can remain in the genome and is passed on to the progeny of the cell when it divides.

### (c) Adenovirus

Adenovirus has been usually employed as a gene delivery system because of its mid-sized genome, ease of manipulation, high titer, wide target-cell range, and high infectivity. Both ends of the viral genome contains 100-200 bp ITRs (inverted terminal repeats), which are cis elements necessary for viral DNA replication and packaging. The E1 region (E1A and E1B) encodes proteins responsible for the regulation of transcription of the viral genome and a few cellular genes. The expression of the E2 region (E2A and E2B) results in the synthesis of the proteins for viral DNA replication.

Of adenoviral vectors developed so far, the replication incompetent adenovirus having the deleted E1 region is usually used. The deleted E3 region in adenoviral vectors may provide an insertion site for transgenes (Thimmappaya, B. et al., Cell, 31:543-551(1982); and Riordan, J. R. et al., Science, 245:1066- 1073 (1989)). Therefore, it is preferred that the decorin-encoding nucleotide sequence is inserted into either the deleted E1 region (E1A region and/or E1B 5 region, preferably, E1B region) or the deleted E3 region. The polynucleotide of the invention may be inserted into the deleted E4 region. The term “deletion” with reference to viral genome sequences encompasses whole deletion and partial deletion as well. In nature, adenovirus can package approximately 105% of the wildtype genome, providing capacity for about 2 extra kb of DNA (Ghosh-Choudhury et al., EMBO J.′ 6:1733-1 739 (1987)). In this regard, the foreign sequences described above inserted into adenovirus may be further 15 inserted into adenoviral wild-type genome.

The adenovirus may be of any of the known serotypes or subgroups A-F. Adenovirus type 5 of subgroup C is the most preferred starting material for constructing the adenoviral gene delivery system of this invention. A great deal of biochemical and genetic information about adenovirus type 5 is known. The foreign genes delivered by the adenoviral gene delivery system are episomal, and genotoxicity to host cells. Therefore, gene therapy using the adenoviral gene delivery system may be considerably safe.

### (d) Adeno-Associated Virus (AAV)

Adeno-associated viruses are capable of infecting non-dividing cells and various types of cells, making them useful in constructing the gene delivery system of this invention. The detailed descriptions for use and preparation of AAV vector are found in U.S. Pat. Nos. 5,139,941 and 4,797,368.

Research results for AAV as gene delivery systems are disclosed in LaFace et al, Viology, 162: 483486 (1988), Zhou et al., Exp. Hematol. (NY), 21:928-933(1993), Walsh et al, J. Clin. Invest., 94:1440-1448(1994) and Flotte et al., Gene Therapy, 2:29-37(1995). Typically, a recombinant AAV virus is made by cotransfecting a plasmid containing the gene of interest (i.e., decorin gene and nucleotide sequence of interest to be delivered) flanked by the two AAV terminal repeats (McLaughlin et al., 1988; Samulski et al., 1989) and an expression plasmid containing the wild type AAV coding sequences without the terminal repeats (McCarty et al., J. Viral., 65:2936-2945(1991)).

### (e) Other Viral Vectors

Other viral vectors may be employed as a gene delivery system in the present invention. Vectors derived from viruses such as vaccinia virus (Puhlmann M. et al., Human Gene Therapy 10:649-657(1999); Ridgeway, “Mammalian expression vectors,” In: Vectors: A survey of molecular cloning vectors and their uses. Rodriguez and Denhardt, eds. Stoneham: Butterworth, 467-492 (1988); Baichwal and Sugden, “Vectors for gene transfer derived from animal DNA viruses: Transient and stable expression of transferred genes,” In: Kucherlapati R, ed. Gene transfer. New York: Plenum Press, 117-148 (1986) and Coupar et al., Gene, 68:1-10(1988)), lentivirus (Wang G. et al., J. Clin. Invest. 104 (11): RS 5-62 (1999)) and herpes simplex virus (Chamber R., et al., Proc. Natl. 10 15 Acad. Sci USA 92:1411-1415(1995)) may be used in the present delivery systems for transferring both the polynucleotide of the invention into cells.

### 6.3.2. CIPN-Inducing Therapeutic Agents

In various embodiments, the neuropathy-inducing therapeutic agent to which the mammal has been exposed is a chemotherapy drug.

In certain embodiments, the chemotherapy drug is a platinum analog. In particular embodiments, the drug is cisplatin, carboplatin, or oxaliplatin. In certain embodiments, the chemotherapy drug is an anti-mitotic agent. In certain embodiments, the drug is a taxane. In particular embodiments, the taxane is paclitaxel (Taxol®), docetaxel (Taxotere®), or cabazitaxel (Jevtana®). In certain embodiments, the drug is eribulin (Halaven®). In certain embodiments, the chemotherapy drug is a plant alkaloid. In particular embodiments, the drug is vinblastine, vincristine, vinorelbine, or etoposide (VP-16). In certain embodiments, the chemotherapy drug is a proteasome inhibitor. In particular embodiments, the drug is bortezomib (Velcade®) or carfilzomib (Kyprolis®). In certain embodiments, the chemotherapy drug is an immunomodulatory agent. In particular embodiments, the drug is thalidomide (Thalomid), lenalidomide (Revlimid®), or pomalidomide (Pomalyst®). In certain embodiments, the chemotherapy drug is an epothilones. In particular embodiments, the drug is ixabepilone) (Ixempra®). In certain embodiments, the neuropathy-inducing therapeutic agent to which the mammal has previously been exposed is an antineoplastic biologic.

The subject includes both non-human mammals and humans exposed or to be exposed to a chemotherapy drug.

### 6.3.3. Order of Administration

In typical embodiments, the nucleic acid construct is administered to a subject who has previously been exposed to the neuropathy-inducing therapeutic agent. In some embodiments, the nucleic acid construct is administered to a subject concurrently with the neuropathy-inducing therapeutic agent. In some embodiments, the nucleic acid construct is administered before the subject is exposed to the neuropathy-inducing therapeutic agent. In some embodiments, the nucleic acid construct is administered both before and after exposure to the chemotherapy drug.

In some embodiments, the method further comprises the step of readministering the DNA to the mammal more than one week after the step of administering. In some embodiments, the step of readministering is done at least 2 weeks, 3 weeks, 4 weeks, 5 weeks, or 10 weeks after the step of administering. In some embodiments, the step of readministering is done at least 10 days, 15 days, 20 days, 30 days, 40 days, 50 days or 100 days after the step of administering. In some embodiments, the mammal is not administered with the nucleic acid construct between the step of administering and the step of readministering.

### 6.3.4. Delivery Methods

Various delivery methods can be used to administer the polynucleotide construct expressing one or more isoforms of HGF in the methods described herein.

### 6.3.4.1. Injection

In typical embodiments, the nucleic acid construct is administered by injection of a liquid pharmaceutical composition.

In currently preferred embodiments, the polynucleotide construct is administered by intramuscular injection. Typically, the polynucleotide construct is administered by intramuscular injection close to the site of pain or patient-perceived site of pain. In some embodiments, the polynucleotide constructs are administered to the muscles of hands, feet, legs, or arms of the subject.

In some embodiments, the construct is injected subcutaneously or intradermally.

In some embodiments, the polynucleotide construct is administered by intravascular delivery. In certain embodiments, the construct is injected by retrograde intravenous injection.

### 6.3.4.2. Electroporation

Transformation efficiency of plasmid DNA into cells in vivo can in some instances be improved by performing injection followed by electroporation. Thus, in some embodiments, the polynucleotide is administered by injection followed by electroporation. In particular embodiments, electroporation is administered using the TriGrid™ Delivery System (Ichor Medical Systems, Inc., San Diego, USA).

### 6.3.4.3. Sonoporation

In some embodiments, sonoporation is used to enhance transformation efficiency of a construct of the present invention. Sonoporation utilizes ultrasound wave to temporarily permeablize the cell membrane to allow cellular uptake of DNA. Polynucleotide constructs can be incorporated within microbubbles and administered into systemic circulation, followed by external application of ultrasound. The ultrasound induces cavitation of the microbubble within the target tissue to result in release and transfection of the constructs.

### 6.3.4.4. Magnetofection

In some embodiments, magnetofection is used to enhance transformation efficiency of a construct of the present invention. The construct is administered after being coupled to a magnetic nanoparticle. Application of high gradient external magnets cause the complex to be captured and held at the target. The polynucleotide construct can be released by enzymatic cleavage of cross linking molecule, charge interaction or degradation of the matrix.

### 6.3.4.5. Liposome

In some embodiments, polynucleotide of the present invention can be delivered by liposomes. Liposomes are formed spontaneously when phospholipids are suspended in an excess of aqueous medium. Liposome-mediated nucleic acid delivery has been very successful as described in Nicolau and Sene, Biochim. Biophys. Acta, 721:185-190(1982) and Nicolau et al., Methods Enzymol., 149:157-176 (1987). Example of commercially accessible reagents for transfecting animal cells using liposomes includes Lipofectamine (Gibco BRL). Liposomes entrapping polynucleotide of the invention interact with cells by mechanism such as endocytosis, adsorption and fusion and then transfer the sequences into cells.

### 6.3.4.6. Transfection

When a viral vector is used to deliver a polynucleotide encoding HGF, the polynucleotide sequence may be delivered into cells by various viral infection methods known in the art. The infection of host cells using viral vectors are described in the above-mentioned cited documents.

Preferably, the pharmaceutical composition of this invention may be administered parenterally. For non-oral administration, intravenous injection, intraperitoneal injection, intramuscular injection, subcutaneous injection, or local injection may be employed. For example, the pharmaceutical composition may be injected by retrograde intravenous injection.

Preferably, the pharmaceutical composition of the present invention may be administered into the muscle. In some embodiments, the administration is targeted to the muscle affected by the neuropathic pain.

### 6.3.5. Dose

The polynucleotide construct is administered in a therapeutically effective dose. In the methods described herein, the therapeutically effective dose is a dose effective to reduce neuropathic pain in the subject.

In some embodiments of the methods described herein, the polynucleotide construct is administered at a total dose of 1 μg to 200 mg, 1 mg to 200 mg, 1 mg to 100 mg, 1 mg to 50 mg, 1 mg to 20 mg, or 5 mg to 10 mg. In some embodiments, the polynucleotide construct is administered at a total dose of 2 mg, 4 mg, 8 mg, 16 mg, 32 mg, or 64 mg.

In various embodiments, the total dose is divided into a plurality of individual injection doses. In some embodiments, the total dose is divided into a plurality of equal injection doses. In some embodiments, the total dose is divided into unequal injection doses. In various divided dose embodiments, the total dose is administered to 4, 8, 16, 24, 32, or 64 different injection sites. In some embodiments, the injection dose per injection site is between 0.1-5 mg. In certain embodiments, the injection dose per injection site is 0.1 mg, 0.15 mg, 0.2 mg, 0.25 mg, 0.3 mg, 0.35 mg, 0.4 mg, 0.45 mg, 0.5 mg or 1 mg.

In typical divided dose embodiments, all of the plurality of injection doses are administered within 1 hour of one another. In some embodiments, all of the plurality of injection doses are administered within 1.5, 2, 2.5 or 3 hours of one another.

In various embodiments of the methods, a total dose of polynucleotide construct, whether administered as a single unitary dose or divided into plurality of injection doses, is administered only once to the subject. In other embodiments, the polynucleotide construct is re-administered several days after the initial administration. In some embodiments, the polynucleotide construct is re-administered about 3, 5, 10, 15, 20, 25, 30, or 35 days after the initial administration. In some embodiments, the polynucleotide construct is re-administered ½, 1, 2, 3, 4, 5, 7, 9, or 10 weeks after the initial administration. In some embodiments, the polynucleotide is re-administered 1, 2, 3, 4, 5, 6, 7, 8, 9, or 10 months after the initial administration. In some embodiments, the subsequent total dose is the same as the initial total dose. In some embodiments, subsequent doses differ from the initial total dose. In some embodiments, the pharmaceutical composition is administered once in two months, once a month, 2-4 times a month, once a week, or once every two weeks.

In some embodiments, administration of a total dose of polynucleotide construct into a plurality of injection sites over one, two, three or four visits can comprise a single cycle. In particular, administration of 32 mg, 16 mg, 8 mg, or 4 mg of polynucleotide construct into a plurality of injection sites over two visits can comprise a single cycle. The two visits can be 3, 5, 7, 14, 21 or 28 days apart.

In some embodiments, the cycle can be repeated. The cycle can be repeated twice, three times, four times, five times, six times, or more. In some embodiments, the cycle can be repeated 1, 2, 3, 4, 5, 6, 7, 8, 9, 10, 11, 12, or more months after the previous cycle.

In some embodiments, the total dose administered in the subsequent cycle is same as the total dose administered in the prior cycle. In some embodiments, the total dose administered in the subsequent cycle is different from the total dose administered in the prior cycle.

In some embodiments, the polynucleotide construct is administered at a dose of 8 mg per affected site (e.g., affected limb), equally divided into a plurality of injections and plurality of visits, wherein each of the plurality of injections in any single visit is performed at a separate injection site. In certain embodiments, the DNA construct is administered at a dose of 8 mg per affected site, equally divided into a first dose of 4 mg per site on day 0 and a second dose of 4 mg per site on day 14, wherein each of the first and second dose is equally divided into a plurality of injection doses. In some embodiments, the administration of 8 mg per affected site can constitute a cycle, and the cycle can be repeated once, twice, three times, or more.

The actual amount administered, and rate and time-course of administration, will depend on the nature and severity of pain being treated. In typical embodiments, the polynucleotide construct is administered in an amount effective to reduce neuropathic pain. In some embodiments, the amount is effective to reduce neuropathic pain within 1 week of administration. In some embodiments, the amount is effective to reduce neuropathic pain within 2 weeks, 3 weeks, or 4 weeks of administration.

In some embodiments, two different types of constructs are administered together to induce expression of two isoforms of HGF, i.e., a first construct encoding flHGF and a second construct encoding dHGF. In some embodiments, a single construct that encodes both flHGF and dHGF is delivered to induce expression of both flHGF and dHGF.

According to the conventional techniques known to those skilled in the art, the pharmaceutical composition may be formulated with pharmaceutically acceptable carrier and/or vehicle as described above, finally providing several forms a unit dose form and a multidose form. Non-limiting examples of the formulations include, but not limited to, a solution, a suspension or an emulsion in oil or aqueous medium, an extract, an elixir, a powder, a granule, a tablet and a capsule, and may further comprise a dispersion agent or a stabilizer.

### 6.3.6. Variations

In vivo and/or in vitro assays may optionally be employed to help identify optimal dosage ranges. The precise dose to be employed in the formulation will also depend on the associated chemotherapy drug, the route of administration, and the seriousness of the condition, and should be decided according to the judgment of the practitioner and each subject's circumstances. Effective doses may be extrapolated from dose-response curves derived from in vitro or animal model test systems.

In some embodiments, the method comprises an additional step of diagnosing CIPN and pain conditions. The diagnosis may involve electromyography with nerve conduction studies, skin biopsies to evaluate cutaneous nerve innervation, and nerve and muscle biopsies for histopathological evaluation.

The polynucleotide construct can be administered alone or in combination with other treatments, either simultaneously or sequentially dependent upon the condition to be treated.

### 6.4. Pharmaceutical Compositions

In typical embodiments, the nucleic acid construct is administered in a liquid pharmaceutical composition.

### 6.4.1. Pharmacological Compositions and Unit Dosage Forms Adapted for Injection

For intravenous, intramuscular, intradermal, or subcutaneous injection, the nucleic acid construct will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In various embodiments, the nucleic acid construct is present in the liquid composition at a concentration of 0.01 mg/ml, 0.05 mg/ml, 0.1 mg/ml, 0.25 mg/ml, 0.5 mg/ml, or 1 mg/ml. In some embodiments, the unit dosage form is a vial containing 2 ml of the pharmaceutical composition at a concentration of 0.01 mg/ml, 0.1 mg/ml, 0.5 mg/ml, or lmg/ml.

In some embodiments, the unit dosage form is a vial, ampule, bottle, or pre-filled syringe. In some embodiments, the unit dosage form contains 0.01 mg, 0.1 mg, 0.2 mg, 0.25 mg, 0.5 mg, 1 mg, 2.5 mg, 5 mg, 8mg, 10 mg, 12.5 mg, 16 mg, 24 mg, 25 mg, 50 mg, 75 mg, 100 mg, 150 mg, or 200 mg of the polynucleotide of the present invention.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 0.25 ml, 0.5 ml, 1 ml, 2.5 ml, 5 ml, 7.5 ml, 10 ml, 25 ml, or 50 ml of pharmaceutical composition.

In particular embodiments, the unit dosage form is a vial containing 1 ml of the pharmaceutical composition at Unit dosage form embodiments suitable for subcutaneous, intradermal, or intramuscular administration include preloaded syringes, auto-injectors, and auto-inject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an auto-inject pen. The auto-inject pen comprises an auto-inject pen containing a pharmaceutical composition as described herein. In some embodiments, the auto-inject pen delivers a predetermined volume of pharmaceutical composition. In other embodiments, the auto-inject pen is configured to deliver a volume of pharmaceutical composition set by the user.

In various embodiments, the auto-inject pen contains about 0.1 mL to about 5.0 mL of the pharmaceutical composition. In specific embodiments, the auto-inject pen contains about 0.5 mL of the pharmaceutical composition. In particular embodiments, the auto-inject pen contains about 1.0 mL of the pharmaceutical composition. In other embodiments, the auto-inject pen contains about 5.0 mL of the pharmaceutical composition.

### 6.4.2. Lyophilized DNA Formulations

In some embodiments, nucleic acid constructs of the present inventions are administered as liquid compositions reconstituted from lyophilized formulations. In specific embodiments, DNA formulations lyophilized as disclosed in U.S. Pat. No. 8,389,492, incorporated by reference in its entirety herein, are used after reconstitution.

In some embodiments, the nucleic acid constructs of the present invention is formulated with certain excipients, including a carbohydrate and a salt, prior to lyophilization. The stability of a lyophilized formulation of DNA to be utilized as a diagnostic or therapeutic agent can be increased by formulating the DNA prior to lyophilization with an aqueous solution comprising a stabilizing amount of carbohydrate.

A carbohydrate of the DNA formulation of the invention is a mono-, oligo-, or polysaccharide, such as sucrose, glucose, lactose, trehalose, arabinose, pentose, ribose, xylose, galactose, hexose, idose, mannose, talose, heptose, fructose, gluconic acid, sorbitol, mannitol, methyl a-glucopyranoside, maltose, isoascorbic acid, ascorbic acid, lactone, sorbose, glucaric acid, erythrose, threose, allose, altrose, gulose, erythrulose, ribulose, xylulose, psicose, tagatose, glucuronic acid, galacturonic acid, mannuronic acid, glucosamine, galactosamine, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, cyclodextrin, pustulan, chitin, agarose, keratin, chondroitin, dermatan, hyaluronic acid, alginic acid, xantham gum, or starch.

In one series of embodiments, the carbohydrate is mannitol or sucrose.

The carbohydrate solution prior to lyophilization can correspond to carbohydrate in water alone, or a buffer can be included. Examples of such buffers include PBS, HEPES, TRIS or TRIS/EDTA. Typically, the carbohydrate solution is combined with the DNA to a final concentration of about 0.05% to about 30% sucrose, typically 0.1% to about 15% sucrose, such as 0.2% to about 5%, 10% or 15% sucrose, preferably between about 0.5% to 10% sucrose, 1% to 5% sucrose, 1% to 3% sucrose, and most preferably about 1.1% sucrose.

A salt of the DNA formulation of the invention is NaC1 or KCl. In certain aspects, the salt is NaCl. In further aspects, the salt of the DNA formulation is in an amount selected from the group consisting of between about 0.001% to about 10%, between about 0.1% and 5%, between about 0.1% and 4%, between about 0.5% and 2%, between about 0.8% and 1.5%, between about 0.8% and 1.2% w/v. In certain embodiments, the salt of the DNA formulation is in an amount of about 0.9% w/v.

The final concentration in liquid compositions reconstituted from lyophilized formulations is from about 1 ng/mL to about 30 mg/mL of plasmid. For example, a formulation of the present invention may have a final concentration of about 1 ng/mL, about 5 ng/mL, about 10 ng/mL, about 50 ng/mL, about 100 ng/mL, about 200 ng/mL, about 500 ng/mL, about 1 μg/mL, about 5μg/mL, about 10 μg/mL, about 50 μg/mL, about 100 μg/mL, about 200 μg/mL, about 400 μg/mL, about 500 μg/mL, about 600 μg/mL, about 800 μg/mL, about 1 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 7 mg/mL, about 8 mg/mL, about 9 mg/mL, about 10 mg/mL, about 20 mg/mL, or about 30 mg mg/mL of a plasmid. In certain embodiments of the invention, the final concentration of the DNA is from about 100 μg/mL to about 2.5 mg/mL. In particular embodiments of the invention, the final concentration of the DNA is from about 0.5 mg/mL to 1 mg/mL.

The DNA formulation of the invention is lyophilized under standard conditions known in the art. A method for lyophilization of the DNA formulation of the invention may comprise (a) loading a container, e.g., a vial, with a DNA formulation, e.g., a DNA formulation comprising a plasmid DNA, a salt and a carbohydrate, where the plasmid DNA comprises an HGF gene, or variant thereof, into a lyophilizer, wherein the lyophilizer has a starting temperature of about 5° C. to about −50° C.; (b) cooling the DNA formulation to subzero temperatures (e.g., −10° C. to −50° C.); and (c) substantially drying the DNA formulation. The conditions for lyophilization, e.g., temperature and duration, of the DNA formulation of the invention can be adjusted by a person of ordinary skill in the art taking into consideration factors that affect lyophilization parameters, e.g., the type of lyophilization machine used, the amount of DNA used, and the size of the container used.

The container holding the lyophilized DNA formulation may then be sealed and stored for an extended period of time at various temperatures (e.g., room temperature to about −180° C., preferably about 2-8° C. to about −80° C., more preferably about −20° C. to about −80° C., and most preferably about −20° C.). In certain aspects, the lyophilized DNA formulations are preferably stable within a range of from about 2-8° C. to about −80° C. for a period of at least 6 months without losing significant activity. Stable storage plasmid DNA formulation can also correspond to storage of plasmid DNA in a stable form for long periods of time before use as such for research or plasmid-based therapy. Storage time may be as long as several months, 1 year, 5 years, 10 years, 15 years, or up to 20 years. Preferably the preparation is stable for a period of at least about 3 years.

The concentration of reconstituted lyophilized DNA in the methods of the current invention is adjusted depending on many factors, including the amount of a formulation to be delivered, the age and weight of the subject, the delivery method and route and the immunogenicity of the antigen being delivered.

### 6.5. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art.

### 6.5.1. Example 1: Preparation of a Nucleic Acid Construct Encoding Isoforms of HGF

Various constructs encoding isoforms of HGF described in U.S. Pat. No. 7,812,146, were used.

In short, the pCK vector was used as a vector capable of expressing isoforms of HGF. The pCK vector is constructed such that the expression of a gene, e.g., an HGF gene, is regulated under enhancer/promoter of the human cytomegalovirus (HCMV), as disclosed in detail in Lee et al., Biochem. Biophys. Res. Commun. 272: 230 (2000); WO 2000/040737, both of which are incorporated by reference in their entirety. Furthermore, cDNA encoding VEGF was cloned into the pCK vector to make pCK-VEGF and E.coli transformed with pCK-VEGF was deposited to Korean Culture Center of Microorganisms on Dec. 27, 1999 (Accession NO: KCCM-10179). The pCK vector includes a polynucleotide of SEQ ID NO: 5. pCK vector has been used for clinical trials on human body, and its safety and efficacy were confirmed (Henry et al., Gene Ther. 18:788 (2011)).

Various sequences encoding isoforms of HGF were used to generate the HGF constructs. In particular, constructs comprising cDNA corresponding exon 1-18 of human HGF and intron 4 of a human HGF gene or its fragment were generated. In the constructs, intron 4 is inserted between exon 4 and exon 5 of the cDNA.

In some cases, the construct comprises a full sequence of intron 4. In some cases, the construct comprises a fragment of intron 4. For example, the construct can contain a nucleotide sequence selected from the group consisting of SEQ ID NO: 7 to SEQ ID NO: 14. The nucleotide sequence of SEQ ID NO: 7 is 7113 bp and corresponds to construct comprising the full sequence of intron 4. The nucleotide sequences of SEQ ID NOS: 8-14 correspond to constructs comprising fragments of intron 4.

Thus, constructs that can be used for the method provided herein have a structure such as: (i) (exon 1 to exon 4)-(nucleotides 483-5438 of SEQ ID NO: 7)-(exon 5 to exon 18); (ii) (exon 1 to exon 4)-(nucleotides 483-2244 nucleotides 3168-5438 of SEQ ID NO: 7)-(exon 5 to exon 18); (iii) (exon 1 to exon 4)-(nucleotides 483-2244 nucleotides 4168-5438 of SEQ ID NO: 7)-(exon 5 to exon 18); (iv) (exon 1 to exon 4)-(nucleotides 483-2244 nucleotides 5117-5438 of SEQ ID NO: 7)-(exon 5 to exon 18) ; (v) (exon 1 to exon 4)-(nucleotides 483-728 nucleotides 2240-5438 of SEQ ID NO: 7)-(exon 5 to exon 18); (vi) (exon 1 to exon 4)-(nucleotides 483-728 nucleotides 3168-5438 of SEQ ID NO: 7)-(exon 5 to exon 18); (vii) (exon 1 to exon 4)-(nucleotides 483-728 nucleotides 4168-5438 of SEQ ID NO: 7)-(exon 5 to exon 18); or (viii) (exon 1 to exon 4)-(nucleotides 483-728 nucleotides 5117-5438 of SEQ ID NO: 7)-(exon 5 to exon 18).

Herein, the hybrid HGF gene including intron 4 of human HGF or its fragment is named “HGF-X”. The HGF-X includes HGF-X1, HGF-X2, HGF-X3, HGF-X4, HGF-X5, HGF-X6, HGF-X7, and HGF-X8 having nucleotide sequences of SEQ ID NO: 7 to SEQ ID NO: 14, respectively. (See TABLE 1 above.)

It was previously demonstrated that two isoforms of HGF (i.e., flHGF and dHGF) can be generated by alternative splicing between exon 4 and exon 5 from each of the constructs. In addition, among the various HGF constructs, HGF-X7 showed the highest level of expression of two isoforms of HGF (i.e., flHGF and dHGF).

HGF-X7 cloned in pCK vector was used for testing efficacy of the treatment methods provided in this Application. As disclosed in U.S. Pat. No. 7,812,146, Escherichia coli Top10F′ transformed with pCK-HGF-X7 was deposited with the accession numbers KCCM-10361, on Mar. 12, 2002.

### 6.5.2. Example 2: Therapeutic Effects of pCK-HGF-X7 on a Mouse Model of Peripheral Neuropathy Induced by Paclitaxel (Taxane)

Paclitaxel (PTX) is a chemotherapy medication sold under the brand name Taxol among others. Paclitaxel is used to treat a number of types of cancer, including ovarian cancer, breast cancer, lung cancer, Kaposi sarcoma, cervical cancer, and pancreatic cancer. Paclitaxel is in the taxane family of medications, working by interference with the normal function of microtubules during cell division. Common side effects of the medication include peripheral neuropathy and neuropathic pain.

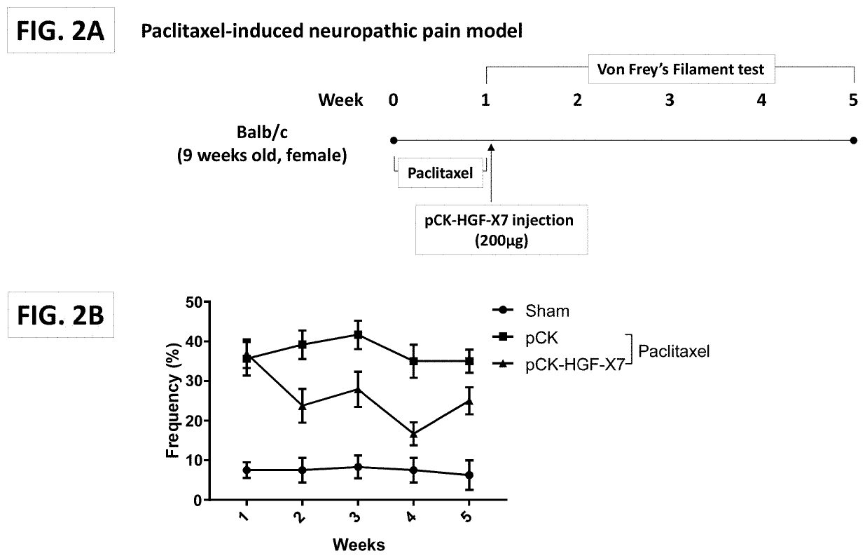

Therapeutic effects of pCK-HGF-X7 on neuropathic pain induced by paclitaxel were studied using a mouse model. As illustrated in FIG. 2A, paclitaxel was administered to 9-week old female Balb/c mice for 1 week on a daily basis through intraperitoneal injections. One week after the start of the chemotherapy injections, the level of allodynia was assessed by Von Frey's filament test and mice exhibiting more than 35% paw withdrawal frequency response were selected as experimental subjects for the study. Sham-treated animals that did not receive chemotherapy agents at week 0 were used as controls.

The experimental animals were administered either (i) 200 μg of pCK-HGF-X7, or (ii) 200 μg of the pCK vector lacking the HGF-X7 payload as a control, by intramuscular injections. Mechanical allodynia was tested weekly for the following 5 weeks. The experimental protocol provided herein is also summarized in FIG. 2A.

As shown in FIG. 2B, sham-treated animals (Sham) exhibited low levels of pain throughout the experiment. Animals treated with paclitaxel, on the other hand, had increased paw withdrawal frequency at week 1, and the paw withdrawal frequency remained high throughout the study period (data not shown). At week 1, animals treated with paclitaxel were divided into two groups, one group injected with pCK-HGF-X7 and the other group injected with pCK vector as a control.

Paw withdrawal frequency of the paclitaxel-treated animals decreased significantly when injected with pCK-HGF-X7, while paw withdrawal frequency did not change when injected with pCK. This result suggests that the animals treated with pCK-HGF-X7 had reduced pain compared to control animals (Sham or pCK). These data suggested that intramuscular administration of pCK-HGF-X7 can have significant pain relieving effects in paclitaxel-induced neuropathic pain.

### 6.5.3. Example 3: Therapeutic Effects of pCK-HGF-X7 on a Mouse Model of Peripheral Neuropathy Induced by Vincristine (Plant Alkaloid)

Vincristine, also known as leurocristine, is a chemotherapy medication sold under the brand name Oncovin, among others. Vincristine is classified as a plant alkaloid. Vincristine is used to treat a number of types of cancer, including acute lymphocytic leukemia, acute myeloid leukemia, Hodgkin's disease, neuroblastoma and small cell lung cancer, among others. Vincristine works partly by binding to the tubulin protein, stopping cells from separating chromosomes during the metaphase. Cells then undergo apoptosis. Vincristine is also known to inhibit leukocyte production and maturation. Common side effects of the medication include neuropathic pain.

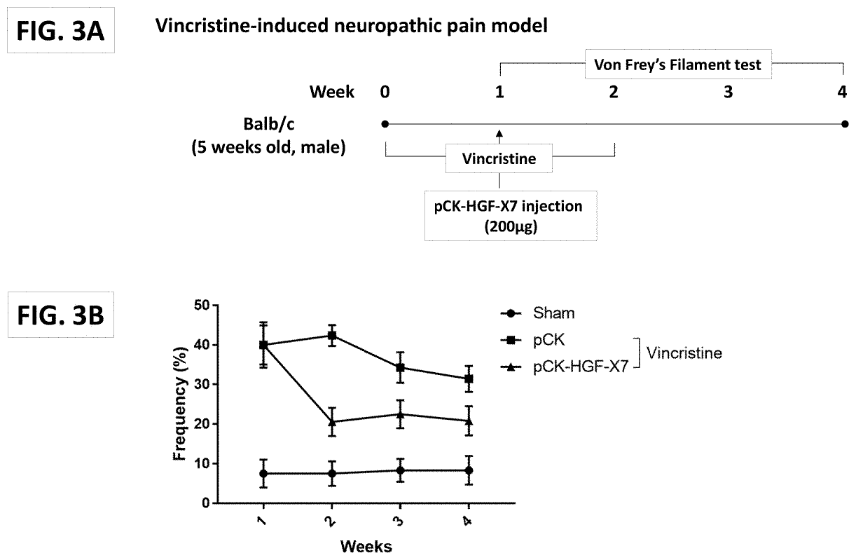

Therapeutic effects of pCK-HGF-X7 on neuropathic pain induced by vincristine were studied using a mouse model. As illustrated in FIG. 3A, vincristine was administered to 5-week old male Balb/c mice for 2 week on a daily basis through intraperitoneal injections. One week after the start of the chemotherapy injections, the level of allodynia was assessed by Von Frey's filament test and mice exhibiting more than 35% paw withdrawal frequency response were selected as experimental subjects for the study. Sham-treated animals that did not receive chemotherapy agents at week 0 were used as controls.

The experimental animals were administered either (i) 200 μg of pCK-HGF-X7, or (ii) 200 μg of the pCK vector lacking the HGF-X7 payload as a control, by intramuscular injections. Mechanical allodynia was tested weekly for the following 4 weeks. The experimental protocol provided herein is also summarized in FIG. 3A.

As shown in FIG. 3B, sham-treated animals (Sham) exhibited low levels of pain throughout the experiment. Animals treated with vincristine, on the other hand, had increased paw withdrawal frequency at week 1, and the paw withdrawal frequency remained high throughout the study period (data not shown). At week 1, animals treated with vincristine were divided into two groups, one group injected with pCK-HGF-X7 and the other group injected with pCK vector as a control.

Paw withdrawal frequency of the vincristine-treated animals decreased significantly when injected with pCK-HGF-X7, while paw withdrawal frequency did not change when injected with pCK. This result suggests that the animals treated with pCK-HGF-X7 had reduced pain compared to control animals (pCK vector only). These data suggested that intramuscular administration of pCK-HGF-X7 has significant pain relieving effects in vincristine-induced neuropathic pain.

### 6.5.4. Example 4: Therapeutic Effects of pCK-HGF-X7 on a Mouse Model of Peripheral Neuropathy Induced by Bortezomib (Proteasome Inhibitor)

Bortezomib is the first therapeutic proteasome inhibitor to be used in humans for the treatment of cancer. Bortezomib is associated with peripheral neuropathy in 30% of patients, accompanied by pain.

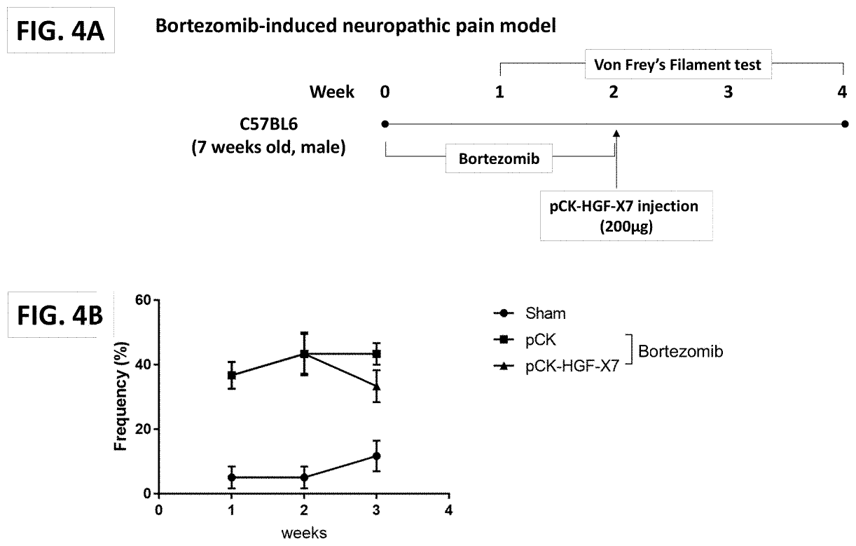

Therapeutic effects of pCK-HGF-X7 on neuropathic pain induced by bortezomib were studied using a mouse model. As illustrated in FIG. 4A, bortezomib was administered to 7-week old male C57BL/6 mice three times a week for 2 weeks through i.p. injections. One week after the start of the chemotherapy injections, the level of allodynia was assessed by Von Frey's filament test and mice exhibiting more than 35% paw withdrawal frequency response were selected as experimental subjects for the study. Sham-treated animals that did not receive chemotherapy agents at week 0 were used as controls.

The experimental animals were administered either (i) 200 μg of pCK-HGF-X7, or (ii) 200 μg of the pCK vector lacking the HGF-X7 payload as a control, by intramuscular injections. Mechanical allodynia was tested weekly, starting one week after the start of the chemotherapy injections. The experimental protocol provided herein is also summarized in FIG. 4A.

As shown in FIG. 4B, sham-treated animals (Sham) exhibited low levels of pain throughout the experiment. Animals treated with bortezomib, on the other hand, showed increased paw withdrawal frequency at week 1, and the paw withdrawal frequency remained high throughout the study period (data not shown). At week 2, animals treated with bortezomib were divided into two groups, one group injected with pCK-HGF-X7 and the other group injected with pCK vector as a control.

Paw withdrawal frequency of the bortezomib-treated animals decreased significantly when injected with pCK-HGF-X7, while paw withdrawal frequency did not change when injected with pCK. These data suggested that intramuscular administration of pCK-HGF-X7 provides significant pain relieving effects in bortezomib-induced neuropathic pain.

### 6.5.5. Example 5: Therapeutic Effects of pCK-HGF-X7 on a Mouse Model of Peripheral Neuropathy Induced by Cisplatin (Platinum Analog)

Cisplatin is a chemotherapy medication used to treat a number of cancers, including testicular cancer, ovarian cancer, cervical cancer, breast cancer, bladder cancer, head and neck cancer, esophageal cancer, lung cancer, mesothelioma, brain tumors and neuroblastoma. Peripheral neuropathy is a serious side effect of cisplatin, although less common compared to other chemotherapy medications.

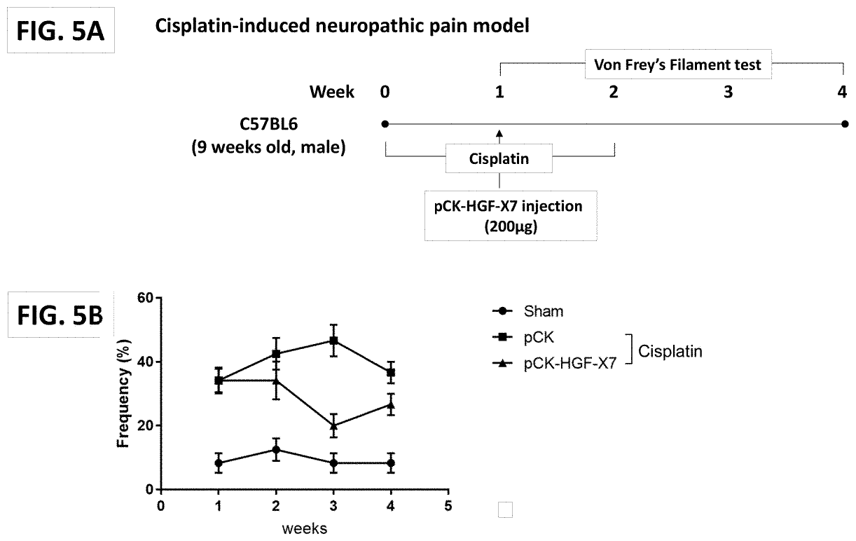

Therapeutic effects of pCK-HGF-X7 on neuropathic pain induced by cisplatin were studied using a mouse model. As illustrated in FIG. 5A, cisplatin was administered to 9-week old male C57BL/6 mice once every two days for 2 week by intraperitoneal injections. One week after the start of the chemotherapy injections, the level of allodynia was assessed by Von Frey's filament test and mice exhibiting more than 35% paw withdrawal frequency response were selected as experimental subjects for the study. Sham-treated animals that did not receive chemotherapy agents at week 0 were used as controls.

The experimental animals were administered either (i) 200 μg of pCK-HGF-X7, or (ii) 200 μg of the pCK vector lacking the HGF-X7 payload as a control, by intramuscular injections. Mechanical allodynia was tested weekly for the following 3 weeks. The experimental protocol provided herein is also summarized in FIG. 5A.

As shown in FIG. 5B, sham-treated animals (Sham) exhibited low levels of pain throughout the experiment. Animals treated with cisplatin, on the other hand, had increased paw withdrawal frequency at week 1, and the paw withdrawal frequency remained high throughout the study period (data not shown). At week 1, animals treated with cisplatin were divided into two groups, one group injected with pCK-HGF-X7 and the other group injected with pCK vector as a control.

Paw withdrawal frequency of the cisplatin-treated animals decreased significantly when injected with pCK-HGF-X7, while paw withdrawal frequency did not change when injected with pCK. These data demonstrate that intramuscular administration of pCK-HGF-X7 has significant pain relieving effects in cisplatin-induced neuropathic pain.

### 7. SEQUENCE

TABLE 2
SEQ ID NO.

| | |
|---|---|
| SEQ ID NO: 1 | Amino acid sequence of flHGF protein |
| SEQ ID NO: 2 | Amino acid sequence of dHGF protein |
| SEQ ID NO: 3 | Nucleotide sequence of exons 1-4 of human hgf |
| SEQ ID NO: 4 | Nucleotide sequence of exons 5-18 of human hgf |
| SEQ ID NO: 5 | Nucleotide sequence of pCK vector |
| SEQ ID NO: 6 | Nucleotide sequence of intron 4 of human hgf |
| SEQ ID NO: 7 | Nucleotide sequence of HGF-X1 |
| SEQ ID NO: 8 | Nucleotide sequence of HGF-X2 |
| SEQ ID NO: 9 | Nucleotide sequence of HGF-X3 |
| SEQ ID NO: 10 | Nucleotide sequence of HGF-X4 |
| SEQ ID NO: 11 | Nucleotide sequence of HGF-X5 |
| SEQ ID NO: 12 | Nucleotide sequence of HGF-X6 |
| SEQ ID NO: 13 | Nucleotide sequence of HGF-X7 |
| SEQ ID NO: 14 | Nucleotide sequence of HGF-X8 |

### 8. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 9. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

1. A method of treating neuropathic pain associated with exposure to a chemotherapy drug, comprising the steps of:
administering to a subject that has previously been exposed to the chemotherapy drug a first therapeutically effective amount of a nucleic acid construct capable of expressing two isoforms of a human hepatocyte growth factor (HGF) protein,
wherein the nucleic acid construct comprises:
a first sequence comprising exons 1-4 of a human HGF gene or a degenerate sequence of the first sequence,
a second sequence comprising intron 4 of the human HGF gene or a fragment of the second sequence, and
a third sequence comprising exons 5-18 of the human HGF gene or a degenerate sequence of the third sequence.

2. The method of claim 1, wherein the chemotherapy drug is selected from the group consisting of a plant alkaloid, a taxane, an epothilone, a proteasome inhibitor, an immunomodulator, and an antineoplastic biologic.

3. The method of claim 2, wherein the chemotherapy drug is vincristine, bortezomib, paclitaxel, or cisplatin.

4. The method of claim 3, wherein the chemotherapy drug is paclitaxel.

5. The method of claim 3, wherein the chemotherapy drug is vincristine.

6. The method of claim 3, wherein the chemotherapy drug is bortezomib.

7. The method of claim 3, wherein the chemotherapy drug is cisplatin.

8. The method of claim 1, wherein the subject is a human cancer patient.

9. The method of claim 1, further comprising the step of readministering the nucleic acid construct to the subject more than one week after the step of administering the first therapeutically effective amount of nucleic acid construct.

10. The method of claim 9, wherein the step of readministering is done at least 2 weeks, 3 weeks, 4 weeks, 5 weeks, or 10 weeks or at least 10 days, 15 days, 20 days, 30 days, 40 days, 50 days or 100 days after the step of administering the first therapeutically effective amount of nucleic acid construct.

11. The method of claim 9, wherein the subject is not administered with the nucleic acid construct between the step of administering the first therapeutically effective amount of nucleic acid construct and the step of readministering.

12. The method of claim 1, wherein the first sequence and the third sequence lack an intron.

13. The method of claim 1, wherein the two isoforms of HGF comprise a full-length HGF (flHGF) and a deleted variant HGF (dHGF).

14. The method of claim 13, wherein the full-length HGF (flHGF) comprises a polypeptide of SEQ ID NO: 1 and the deleted variant HGF (dHGF) comprises a polypeptide of SEQ ID NO:2.

15. The method of claim 1, wherein the first sequence comprises a polynucleotide of SEQ ID NO: 3.

16. The method of claim 1, wherein the second sequence comprises a polynucleotide of SEQ ID NO: 6 or a fragment thereof.

17. The method of claim 1, wherein the third sequence comprises a polynucleotide of SEQ ID NO: 4.

18. The method of claim 1, wherein the nucleic acid construct comprises a polynucleotide of SEQ ID NO: 13.

19. The method of claim 1, wherein the nucleic acid construct is VM202.

20. The method of claim 1, wherein the step of administering the first therapeutically effective amount of nucleic acid construct or the step of readministering comprises one or more intramuscular injections of the nucleic acid construct.

21. The method of claim 1, wherein the first therapeutically effective amount of nucleic acid construct is between 1 μg and 100 mg, between 10 μg and 50 mg, between 100 μg and 10 mg, between 1 mg and 25 mg, or between 1 mg and 10 mg.


SEQUENCE LISTING (14 sequences)

SEQ ID NO: 1 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 2 - protein, 723 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 3 - DNA, 482 bp, Homo sapiens
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacag

SEQ ID NO: 4 - DNA, 1675 bp, Homo sapiens
cctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 5 - DNA, 3756 bp, Artificial Sequence
  Description of Artificial Sequence Synthetic polynucleotide
cgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 6 - DNA, 4956 bp, Homo sapiens
gtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaaga

SEQ ID NO: 7 - DNA, 7113 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 8 - DNA, 6190 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 9 - DNA, 5190 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatcctgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 10 - DNA, 4241 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccttatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 11 - DNA, 5602 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 12 - DNA, 4679 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 13 - DNA, 3679 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatcctgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 14 - DNA, 2730 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatccttatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag